(19) United States
(12) Patent Application Publication
(10) Pub. No.: US 2026/0283813 A1
(43) Pub. Date: Sep. 24, 2026

(54) AN IMPLANT DEVICE FOR TOTAL ANKLE REPLACEMENT

(71) Applicant: Ketankumar JAJAL, Vadodara, Gujarat (IN)

(72) Inventors: Ketankumar JAJAL, Vadodara, Gujarat (IN); Rajiv SHAH, Vadodara, Gujarat (IN); Ravikant KAMAL, Vadodara, Gujarat (IN); Jay PRAJAPATI, Chitrakoot, Gujarat (IN); Chirag RAMANI, Ahmedabad, Gujarat (IN)

(21) Appl. No.: 19/472,719

(22) PCT Filed: Apr. 12, 2024
(86) PCT No.: PCT/IN2024/050391

§ 371 Date: Oct. 6, 2025

(30) Foreign Application Priority Data
Apr. 12, 2023 (IN) 202321027156

(51) Int. Cl. A61F 2/42

(52) U.S. Cl. CPC A61F 2/4202; A61F 2002/4205; A61F 2002/4207

(57) ABSTRACT

The apparatus for total ankle replacement relates to a 3-component system for replacing the entire ankle joint comprising a tibia implant 100 for replacing a deformed tibia bone, a talus implant 200 for replacing a deformed talus implant and a mobile structure sliding interface 300 sandwiched between the said tibia implant and the talus implant to enable the relative motion between the said tibia implant 100 and the talus implant 200 in order to restore the natural movement of an ankle joint.

### FIELD OF INVENTION

The present invention relates to the field of orthopaedic implants. More particularly the present invention relates to an implant device for total ankle replacement which is customised for each ankle joint.

### BACKGROUND OF INVENTION

The ankle is a large joint made up of three bones:
The shin bone (tibia)
The thinner bone running next to the shin bone (fibula)
A foot bone that sits above the heel bone (talus)

The bony bumps (or protrusions) seen and felt on the ankle have their own names:
The medial malleolus, felt on the inside of your ankle is part of the tibia's base
The posterior malleolus, felt on the back of your ankle is also part of the tibia's base
The lateral malleolus, felt on the outside of your ankle is the low end of the fibula

The ankle joint allows up-and-down movement of the foot. The subtalar joint sits below the ankle joint, and allows side-to-side motion of the foot. Numerous ligaments (made of tough, moveable tissue) surround the true ankle and subtalar joints, binding the bones of the leg to each other and to those of the foot.

The ankle replacement is carried out to provide pain relief while preserving ankle motion, so the patient has less pain and better function during activity.

The first generation of Total Ankle Replacement (TAR) devices were formed by two components, a concave polyethylene tibial component and a convex metal (usually cobalt-chrome alloy) talar component. Constrained or unconstrained TAR designs were introduced, but poor results and high failure rates were recorded with both types. With constrained implants, the inability to dissipate the rotational forces produced by the continuous variation of rotational axis resulted in loosening as the main cause of failure. On the other hand, with unconstrained designs, instability occurred due to the excessive strain placed on surrounding soft tissues. First-generation implants all required large bone resection to allow cement fixation and component positioning. This has been shown to be another drawback and possible cause of loosening of these devices for two main reasons. In summary, first-generation implants were abandoned because of the high failure rates, most commonly due to cement fixation, over-constraint, lack of constraint, wound healing, component loosening and pain.

US Patent Publication No. U.S. Pat. No. 40,69,518A discloses one such type of device. The document discloses a prosthetic joint for the replacement of the ankle joint comprising a tibial member and a talar member each having three distinct complementary bearing surfaces which allow plantar and dorsal flexion and some rotation approximating the movement of the natural ankle joint. The talar member has three adjacent bearing surfaces which are each longitudinally and laterally convexly shaped and the tibial member is provided with three substantially complementary longitudinally and laterally concavely shaped bearing surfaces that provide medial-lateral support while allowing about 5° rotation between the members.

French Patent Publication No. FR2724108A1 discloses one such type of device. The document discloses a prosthesis consists of first and second components for attachment to the respective bone ends, having complementary curved surfaces which come into sliding contact with each other. The curved surfaces are formed as one convex and one concave surface, with a central rib and groove forming a stabilising connection between the two components. The rib and groove define the sliding surfaces as a pair of parallel part-toroidal surfaces. At least one of the components is formed, on the opposite side to its curved sliding surface, with one or more anchor plates for engagement in a bone surface. The anchor plates have serrated formations along their upper edge surfaces.

The second phase of implants began with the introduction of modern TARs, like the Buechel-Pappas Total Ankle Replacement (Endotec, South Orange, NJ) in the USA and the Scandinavian Total Ankle Replacement (STAR; Waldemar Link, Hamburg, Germany) in Europe. The Agility Total Ankle System prosthesis (DePuy, Warsaw, IN), designed by Dr. Frank Alvine, was the first ankle implant to receive FDA approval. At the end of this phase, almost all TARs were semi-constrained, cementless (with minimal bone resection required), and using porous coatings to encourage bone ingrowth. Tibial metal-backed, polyethylene inserts and large contact areas on the tibia and talus became common features as well.

The last phase introduction of a few new implants, including the Salto (Torn-ier SA, Saint Ismier, France), HINTEGRA (Newdeal SA, Lyon), Mobility (DePuy, Warsaw, IN), TNK (Kyocera Corporation, Japan), and BOX (Finsbury Orthopaedics, Leatherhead, Surrey, UK). All these designs (except for the TNK which is a ceramic implant) use the three-part mobile bearing system.

US Patent Publication No. US2010035019A1 discloses a total ankle replacement system is presented. The total ankle joint replacement procedure can be used to treat persons with disability, deformity, or that are suffering from osteoarthritis and other arthritic conditions. The total ankle joint assembly generally comprises a tibial component, a talar component, and a bearing component, where the bearing component is positioned between and articulates with the tibial component and talar component to mimic the natural ankle joint movement.

US Patent Publication No. U.S. Pat. No. 9,681,958B2 discloses an endoprosthesis for replacing the ankle joint includes a lower component which is configured to be connected to the ankle bone, an upper component which is configured to be connected to the shin bone, and an intermediate part which forms a slide joint both with the lower and upper components. The intermediate part, which is wedge-shaped in sagittal section, is provided in order to compensate for anatomical or surgical irregularities. The upper component can also be wedge-shaped in frontal or sagittal section.

US Patent Publication No. U.S. Pat. No. 11,013,607B2 discloses a talar component of an ankle joint prosthesis for engagement with a talus bone having a medial side wall and a lateral side wall, opposite the medial side wall, each side wall terminating at a distal edge, and the distal edges adapted to drive into the talus bone. When implanted, the side walls may form a seal between the talus bone and the component to prevent fluid from flowing under the component.

US Patent Publication No. U.S. Pat. No. 8,668,743B2 discloses an orthopedic prosthesis, system and method has a dual bearing component that, along with first and second bone anchoring components, provides multi-axial movement separately with respect to both the first and second bone anchoring components. An ankle prosthesis, system and method may thus be fashioned utilizing these principles that includes a dual bearing component, a tibial component adapted for attachment to the tibia bone, and a talar component adapted for attachment to the talus or calceneus bone of the foot. The dual bearing component includes a superior bearing providing gliding articulation/translation between it and the tibial component, and an inferior bearing providing gliding articulation/translation between it and the talar component. A bearing component plate provides a base or foundation for the superior and inferior bearings. The superior bearing is bonded to the bearing component plate while the inferior bearing moves with respect to the bearing component plate.

US Patent Publication No. U.S. Pat. No. 6,852,130B2 discloses endoprosthesis for replacement of the ankle joint includes a component which is to be connected to the anklebone and which forms an upper slide surface a component which is to be connected to the tibia and which forms a lower slide surface, and a middle part. The middle part forms two slide surfaces which interact with slide surfaces on the tibial component and the anklebone component. In frontal section, the middle part is wedge-shaped with a wedge angle of between 1° and 12°.

US Patent Publication No. US20050049711A1 discloses an Ankle implant systems and methods are provided that can allow a surgeon to select the type of prosthesis desired during an ankle surgical operation. The surgeon can implant a set of standardized fixation components into the tibia and/or fibula bones and the talus bone. Once implanted, the surgeon can select a bearing component from a number of bearing components that allow for different size patients, but also modify the manner in which the prosthesis functions (either semiconstrained or unconstrained). In one embodiment, an ankle implant can include a talar component having a lower surface with a bone fixation portion for fixation to a talus bone. A tibial component has an upper surface with a bone fixation portion for fixation of the tibial component to a tibia bone and/or a fibula bone, and the tibial component also has a lower surface with at least one protrusion extending from the lower surface. A bearing component is included between the tibial and talar components and has a lower surface for cooperative engagement with an upper surface of the talar component. The protrusion of the tibial component is adapted to engage a recess of the bearing component to desirably limit rotational and translational movement of the tibial component relative to the bearing component.

The total ankle system by Hintermann is also a three-component system having a tibia component, a talus component and a poly component between the said tibia component and the talus component. The total ankle system by Hintermann is illustrated in the FIG. 1 where it can be seen that the tibia component has standardised cross-section with straight edges. The said straight edges lead to the impingement of the fibula bone. Further, additional screws are required to be inserted into the bone in order to affix the implant to the ankle. Moreover, the talus component has standardised sidewalls for the affixation with the talus bone. The side walls are provided to prevent the dislocation of the poly. The said side walls necessitate the requirement of additional resection from the sides of the talus bone.

The present invention overcomes the issues of the Hinterman total ankle system by providing a total ankle replacement system which have integrated cylindrical pegs with the tibia component and the talus component. The said integrated pegs insert into the tibia bone and the talus bone for affixation. Therefore, additional screws are not required for the said affixation thus reducing the total bone resection. Further, in the present invention, the peripheral edge of the tibia implant is a customised tibial peripheral edge 104 which is customised as per the specific patient to match the cross-sectional peripheral layout of the tibia bone therefore preventing the impingement of the fibula by overhanging edges. In the present invention, the talus component does not have raised sidewalls thus preventing the requirement of additional resection from the sides of the talus bone. To stabilise the poly sliding interface of the present invention, the tibia component is provided with a ridge which mates with a complementary groove in the sliding interface.

Another popular ankle replacement system is the total ankle system by STAR ankle. As illustrated in the FIG. 2, the said system is a three-component system comprising a tibia component, a talus component and a poly component between them. In the said STAR ankle system the peripheral edges of the tibia component are straight as per the standard design. This creates an overhang of the excess material and leads to the impingement of the fibula bone. Here, the talus component is provided with side walls for stabilisation on the talus bone which demands additional resection of the talus bone. The talus component of the STAR ankle system is provided with a ridge for preventing the dislocation of the poly in the longitudinal direction. Further, the tibia component of the said system is provided with horizontal The present invention employs a tibia component whose peripheral edge is designed in a customised way to match the cross-sectional periphery of the tibia of the patient so as to avoid any overhang which could lead to the impingement of the fibula bone. In the present total ankle replacement apparatus, the talus implant is provided with two guiding ridges which guide the sliding movement between the talus implant and the sliding interface. This also provides additional protection against the dislocation of the sliding interface in the lateral direction. The present invention also provides a stopper ridge in the middle of the said guiding ridges which prevent the dislocation of the sliding interface in the longitudinal direction.

### SUMMARY OF THE INVENTION

The primary object of the apparatus for total ankle replacement is to provide a three-part mobile ankle replacement system. In the said three-component system, the first part is a tibia component which is meant for the replacement of the deformed part of the tibia bone which forms the ankle joint. Further a talus component is provided which for the replacement of the deformed part of the tibia bone which forms a part of the ankle joint. The third component is located between the said tibia component and the said talus component. The said third component is not rigidly attached to any bone or any of the said tibia and talus implants thus is located in a mobile manner.

A central aspect of the present total ankle replacement system is to allow for the natural ankle movement. The said movement is enabled by the three-part system wherein two rigidly fixed components are interface by a mobile component which allows the relative movement between the said two rigidly fixed components.

Another aspect of the present invention is to provide a combination of ridges on the rigid components, i.e., the tibia implant and the talus implant, and complementary grooves on the mobile component, i.e., the sliding interface, which ensure that when the rigid components and the mobile component are assembled, the complementary ridges and the grooves are mated which allow the relative sliding movement without the dislocation of the said sliding interface from the intended position between the tibia implant and the talus implant.

Yet another central aspect of the apparatus for total ankle replacement is to provide the tibia implant having a peripheral layout which matches the cross-sectional periphery la of the tibia bone 1 at the resected part. Similarly, the talus implant has a peripheral layout which matches the cross-sectional periphery of the tibia bone at the resected part. The patient-specific customisation of the peripheral layout of the said implants prevents any overhanging material which may lead to any impingement.

The said tibia implant and the talus implant are provided with cylindrical projections at their respective surfaces which are to be attached to the resected tibia bone and the talus bone. This eliminates the requirement of screws for the affixation of the implant with the bones thus reducing the total resection of the bones.

The surface, tibia mating surface, of the tibia implant which is to be fastened to the resected face of the tibia bone is provided with at least one cylindrical projection, tibia peg which is positioned at an angle with respect to the said tibia mating surface. The tibial ankle surface which is the surface of the tibia implant which is to be in contact with the sliding interface is preferably flat and it provided with a tibia stopper ridge at the rear edge which prevents the dislocation of the sliding interface.

Similarly, the surface, talus mating surface, of the tibia implant which is to be fastened to the resected face of the talus bone is provided with at least one cylindrical projection, talus peg which is positioned at an angle with respect to the said talus mating surface. The talus ankle surface which is the surface of the tibia implant which is to be in contact with the sliding interface is a convexly curved surface to enable the dorsiflexion and plantarflexion motions of the ankle. The said curved talus ankle surface is provided with a at least one guiding ridge which guides the motion of the sliding interface on the talus implant along he curved surface. Further, the talus ankle surface is provided with a stopper ridge which limit the maximum relative motion between the talus implant and the sliding interface thus preventing the dislocation of the sliding interface.

Moreover, in the various 3-component solutions for total ankle replacement defined by the prior art, such as the US publication U.S. Pat. No. 11,369,481, the intermediate part is rigidly fixed to the tibia component, whereas the present invention provides dual-mobility by having the intermediate component be completely mobile with respect to the tibia component and as well as the talus component therefore providing a much higher range of motion.

The sliding interface is sandwiched between the tibial ankle surface and the talus ankle surface wherein the surface facing the tibia bone, tibia face, is flat in order to be complementary to the flat tibial ankle surface of the tibia implant. Similarly, the talus face of the sliding interface, which is the side of the sliding interface in contact with the talus ankle surface, is concavely shaped and contains grooves, including the guiding groove and the stopper groove, which are negative impressions of the guiding ridges and the talus stopper ridge respectively.

The tibia implant 100 of an embodiment of the present invention is composed of the tibia implant 100 having at least one angularly positioned cylindrical projection tibia peg 103 on a tibia mating surface 101, a tibial ankle surface 102 of the said tibia implant 100 is provided with a raised lip tibia stopper ridge 105 on its rear edge for the prevention of the dislocation of a sliding interface 300.

The talus implant 200 of an embodiment of the present invention is composed of the talus implant 200 having at least one angularly positioned cylindrical projection talus peg 203 on a talus mating surface 201, a talus ankle surface 202 is provided with at least one raised projection guiding ridge 204 running along the entire anterior-posterior length of the said talus implant 200 and a raised projection talus stopper ridge 205 running along the partial anterior-posterior length of the said talus implant 200.

The sliding interface 300 of an embodiment of the present invention is composed of an unattached mobile structure sliding interface 300 positioned between the tibial ankle surface 102 and the talus ankle surface 202 in a sandwiched manner having a surface tibia face 301 of the sliding interface 300 complementing the tibial ankle surface 102 of the tibia implant 100, a surface talus face 302 of the sliding interface 300 having at least one negative impression of the guiding ridges 204 of the talus implant 200 as a guiding groove 303 and a negative impression of the talus stopper ridge 205 of the talus implant 200 as a stopper groove 304.

### BRIEF DESCRIPTION OF DRAWINGS

The novel features and characteristics of the disclosure are set forth in the description. The disclosure itself, however, as well as a preferred mode of use, further objectives and advantages thereof, will best be understood by reference to the following description of an illustrative embodiment when read in conjunction with the accompanying drawings. One or more embodiments are now described, by way of example only, with reference to the accompanying drawings wherein like reference numerals represent like elements and in which:

FIG. 1: Illustration of the Hintermann ankle system (prior art)

FIG. 2: Illustration of the STAR Ankle system (prior art)

FIG. 3: Illustration of an ankle implanted with the apparatus for total ankle replacement

FIG. 4: Illustration of the various landmarks on the ankle joint

FIG. 5: Illustration of the various axes of the ankle joint

FIG. 6: Illustration of the various planes of the ankle joint

FIG. 7: Illustration of a tibia bone with a deformity

FIG. 8: Illustration of the resection plan of the deformed tibia bone

FIG. 9: Illustration of the cross-sectional edge of the resected tibia bone

FIG. 10: Illustration affixation of the tibia implant on the resected tibia bone

FIG. 11: Illustration of the matching of the peripheral edge of the tibia implant to the cross-section edge of the tibia bone

FIG. 12: Illustration of an embodiment of the tibia implant having a curved tibial ankle surface

FIG. 13: Illustration of the tibia implant having smoothened edges

FIG. 14: Illustration of the top view of the tibia implant

FIG. 15: Illustration of the bottom view of the tibia implant

FIG. 16: Illustration of the resection plan of the talus bone

FIG. 17: Illustration of the resected talus bone

FIG. 18: Illustration of the side view of the talus bone with the talus implant

FIG. 19: Illustration of the isometric view of the talus bone with the talus implant

FIG. 20: Illustration of the curvature of the guiding ridge

FIG. 21: Illustration of the curvature of the talus stopper ridge

FIG. 22: Illustration of the cross-sectional edge of the resected talus bone

FIG. 23: Illustration of the talus implant having a customised talus peripheral edge matching the cross-sectional edge of the resected talus bone

FIG. 24: Illustration of the side view of the talus implant

FIG. 25: Illustration of the top view of the talus implant

FIG. 26: Illustration of the 2D curvature of the talus face of the sliding interface

FIG. 27: Illustration of the 3D curvature of the talus face of the sliding interface

FIG. 28: Illustration of the formation of the guiding grooves of the sliding interface

FIG. 29: Illustration of the formation of the stopper groove of the sliding interface

FIG. 30: Illustration of the sliding interface with smoothened edges

FIG. 31: Illustration of the top view of the sliding interface

FIG. 32: Illustration of an embodiment of the sliding interface having a convex tibia face to match a tibia implant having a concave tibial ankle surface

FIG. 33: Illustration of the assembly of the talus implant and the sliding interface

FIG. 34: Illustration of an ankle having the apparatus for total ankle replacement in a neutral position

FIG. 35: Illustration of the ankle having the apparatus for total ankle replacement in a dorsiflexion position

FIG. 36: Illustration of an ankle having the apparatus for total ankle replacement in a plantarflexion position

FIG. 37: Illustration of the Apparatus for total ankle replacement The figures depict embodiments of the disclosure for purposes of illustration only. One skilled in the art will readily recognize from the following description that alternative embodiments of the assemblies, structures and methods illustrated herein may be employed without departing from the principles of the disclosure described herein.

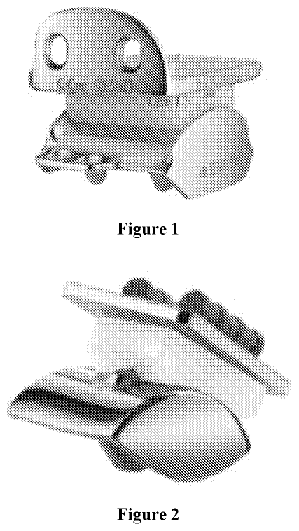

### DETAILED DESCRIPTION OF THE INVENTION

For the purpose of promoting, an understanding of the principles of the invention, references will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof

Reference herein to “one embodiment” or “another embodiment” means that a particular feature, structure, or characteristics described in connection with the embodiment can be included in at least one embodiment of the invention. The appearances of the phrase “in one embodiment” in various places in a specification are not necessarily all referring to the same embodiment, nor are separate or alternative embodiments mutually exclusive of other embodiments. Further, the diagrams representing one or more embodiments of the invention do not inherently indicate any particular order nor imply any limitations in the invention.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more sub-systems or elements or structures or components preceded by “comprises ... a” does not, without more constraints, preclude the existence of other, sub-systems, elements, structures, components, additional sub-systems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this invention belongs. The system, methods, and examples provided herein are only illustrative and not intended to be limiting.

Embodiments of the present invention will be described below in detail with reference to the accompanying figures.

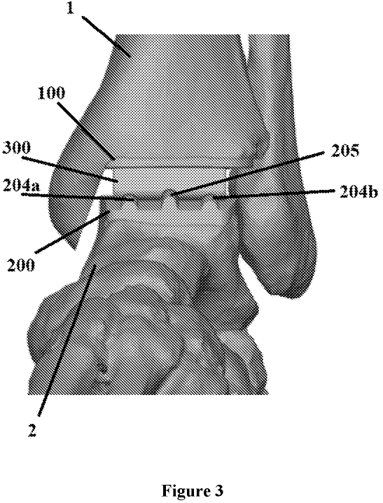

The implant device for total ankle replacement comprises three parts. An embodiment of the present invention is illustrated by the FIG. 3. The first component being a tibia implant 100, the second component being a talus implant 200 and the third part being a sliding interface 300 present between the said tibia implant 100 and the said talus implant 200.

The first part, i.e., the tibia implant 100 is affixed to the part of the tibia bone interfacing with the talus bone. The talus bone is also known as the ankle bone. The second component, i.e., the talus implant 200 is affixed to the part of the talus bone mating with the tibia bone. The sliding interface 300 is positioned between the said tibia implant 100 and the said talus implant 200. The sliding interface 300 is movable with respect to both the tibia implant 100 and the talus implant 200. This enables the relative motion between the tibia implant 100 and the talus implant 200, thus restoring motion to the replaced ankle.

The present invention improves upon the prior art by provided a dual-mobile apparatus. The tibia implant 100 is rigidly fixed to the tibia bone 1 and the talus implant 200 is rigidly fixed to the talus bone 2 with the sliding interface 300 sandwich between the said tibia implant 100 and the talus implant 200 in a completely mobile manner, therefore the sliding interface 300 is movable with respect to the tibia implant 100 and as well as with respect to the talus implant 200 thus providing dual-mobility.

The tibia implant 100 is preferably a monolithic structure. The term monolithic refers to a structure being an undifferentiated and often rigid whole. The tibia implant 100 is singular structure without any joints or seams. In the preferred embodiment of the present invention, the tibia primarily comprises of a tibial mating surface 101 and a tibial ankle surface 102. When the apparatus for total ankle replacement is implanted, the tibial mating surface 101 is mated with the resected end of the tibia bone. At least one elongated projection, tibia peg 103, jutting outwards from the tibial mating surface 101 of the tibia implant 100. The said peg inserts into the resected part of the tibia bone in order to stabilise the position 2 of the tibia implant 100 with respect to the tibia bone.

The said projection tibia peg 103 is at an angle with respect to the tibial mating surface 101 of the tibia implant 100, i.e., the peg is not perpendicular with respect to the tibial mating surface 101 of the tibia implant 100. The said peg being at an angle prevents any anterior or rotational motion with respect to the tibia bone. In the preferred embodiment of the present invention, two tibia pegs 103a & 103b are provided on the tibial mating surface 101 of the tibia implant 100 to provide for higher stability against anterior or rotational motion. The said two tibia pegs 103a & 103b make the same angle with the tibial mating surface 101 of the tibia implant 100 and are parallel to each other. In the preferred embodiment the pegs are of circular cross-section. Other embodiments of the present invention may provide greater numbers of pegs.

The tibial ankle surface 102 of the tibia implant 100 interacts with the sliding interface 300 of the present apparatus. The rear edge of the tibial ankle surface 102 of the tibia implant 100 is provided with a tibia stopper ridge 105. The tibia stopper ridge 105 is a raised lip running along the length of the rear edge of the lower surface of the tibia implant 100 wherein the said ridge projects perpendicular to the said tibial ankle surface 102 of the tibia implant 100. In the preferred embodiment of the present invention, the thickness of the tibia stopper ridge 105 is in the range 0.8-2.5 mm with the most preferred thickness for the said tibia stopper ridge 105 being 1.5 mm When the apparatus for total ankle replacement is implanted at the intended site, the sliding interface 300 forms the movable interface between the tibia implant 100 and the talus implant 200. The tibia stopper ridge 105 of the tibia implant 100 prevents the sliding interface 300 from getting dislocated from its intended position between the tibia implant 100 and the talus implant 200 while there is relative movement between the said tibia implant 100 and the talus implant 200.

In the preferred embodiment of the present invention, the lower surface of the tibia implant 100 is completely flat. In another embodiment of the present invention, the tibial ankle surface 102 of the tibia implant 100 is provided with a slight curve which makes the tibial ankle surface 102 of the tibia implant 100 concave in shape. The flat profile of the tibial ankle surface 102 of the tibia implant 100 helps in restoring the natural movement of the ankle joint by allowing relative sliding motion between the tibial ankle surface 102 and the sliding interface 300. The said motion between the tibial ankle surface 102 and the sliding interface 300 allows for an enhanced range of motion of the ankle.

The tibia implant 100 of an embodiment of the present invention is composed of the tibia implant 100 having at least one angularly positioned cylindrical projection tibia peg 103 on a tibia mating surface 101, a tibial ankle surface 102 of the said tibia implant 100 is provided with a raised lip tibia stopper ridge 105 on its rear edge for the prevention of the dislocation of a sliding interface 300.

The talus implant 200 is affixed on the resected end of the talus bone. In the preferred embodiment of the present invention, the talus implant 200 as well is a monolithic structure, i.e., it is a singular structure with no joints or seams. The talus mating surface 201 of the talus implant 200 is the surface which is affixed to the talus bone. The talus mating surface 201 of the talus implant 200 has an overall concave profile to mate with the talus bone which has been resected to form a convex profile. The said concave talus mating surface 201 of the talus implant 200 contains an elongated projection, talus peg 203, and jutting outwards from the joining surface of the talus implant 200. The said peg inserts into the resected part of the talus bone in order to stabilise the position of the talus implant 200 with respect to the talus bone.

The said talus peg 203 is at an angle with respect to the talus mating surface 201 of the talus implant 200 i.e., the talus peg 203 is not at a 90° with respect to the talus mating surface 201 of the talus implant 200. The said talus peg 203 being at an angle prevents any anterior or rotational motion with respect to the talus bone. In the preferred embodiment of the present invention, one peg is provided on the talus mating surface 201 of the talus implant 200 to provide for stability against anterior or rotational motion. In the preferred embodiment the pegs are of circular cross-section. Other embodiments of the present invention may provide greater numbers of talus peg 203s.

The surface of the talus implant 200, talus ankle surface 202, which remains in mobile joining with the sliding interface 300 has an overall convex profile. The said convex profile of the talus ankle surface 202 of the talus implant 200 is provided in order to facilitate the natural movement of the ankle joint, especially the dorsiflexion and the plantarflexion movements of the ankle joint.

The talus ankle surface 202 of the talus implant 200 is provided with a radially located guiding ridge 204 running along in a radial manner. The said guiding ridge 204 is a raised part jutting radially outward from the talus ankle surface 202 of the talus implant 200. In the preferred embodiment of the present invention, the said guiding ridge 204 runs along the entire anterior-posterior length length of the talus ankle surface 202. In the said embodiment, a guiding ridge 204 is provided close to each of the two lateral side edges of the talus implant 200.

In the preferred embodiment, the said two radially located guiding ridges 204a & 204b are parallel to each other. In the said embodiment, the distance of a guiding ridge 204 from its respective closer lateral edge of the talus implant 200 is equal for both the guiding ridges 204a & 204b. Thus, the said two guiding ridges 204a & 204b are positioned radially and symmetrically on the talus ankle surface 202 of the talus implant 200.

The talus ankle surface 202 is further provided with a talus stopper ridge 205. The said talus stopper ridge 205 is a radially located ridge positioned between the two guiding ridges 204a & 204b. The talus stopper ridge 205 runs parallel to the said two guiding ridges 204a & 204b. In the preferred embodiment, the talus stopper ridge 205 is present in the middle of the said two guiding ridges 204a & 204b, equidistant from the said two guiding ridges 204a & 204b. The length of the talus stopper ridge 205 is less than the length of the guiding ridges 204a & 204b.

The talus implant 200 of an embodiment of the present invention is composed of the talus implant 200 having at least one angularly positioned cylindrical projection talus peg 203 on a talus mating surface 201, a talus ankle surface 202 is provided with at least one raised projection guiding ridge 204 running along the entire anterior-posterior length of the said talus implant 200 and a raised projection talus stopper ridge 205 running along the partial anterior-posterior length of the said talus implant 200.

Between the said tibia implant 100 and the talus implant 200, a sliding interface 300 is located in a sandwiched manner. The sliding interface 300 is a monolithic structure, i.e., the said sliding interface 300 is a singular structure with no joints or seams on its body. The sliding interface 300 has a design such that it is not rigidly attached to either the tibia implant 100 or the talus implant 200. This allows for the relative motion between the said parts. The said sliding interface 300 has two surfaces, the first being the tibia face 301 which remains in contact with the tibial ankle surface 102 of the tibia implant 100, and a second surface being the talus face 302 which remains in contact with the talus ankle surface 202.

The tibia face 301 of the sliding interface 300 is designed to have a profile complementary to the profile of the tibial ankle surface 102 of the tibia implant 100 and the talus face 302 of the sliding interface 300 has a profile complementary of the profile of the talus ankle surface 202 of the talus implant 200. This design keeps the sliding interface 300 engaged with tibia implant 100 and the talus implant 200 while ensuring, the said sliding interface 300 does not get dislocated from its intended position between the tibia implant 100 and the talus implant 200. On the talus face 302 of the sliding interface 300, grooves are provided which are complementary in shape to the guiding ridges 204a & 204b and the talus stopper ridge 205. The said complementary grooves comprise of guiding grooves 303a & 303b which are negative impressions of the guiding ridges 204a & 204b on the talus ankle surface 202. Similarly, the stopper groove 304 present on the talus face 302 of the sliding interface 300 is a negative impression of the talus stopper ridge 205 on the talus ankle surface 202. Therefore, when the talus implant 200 an the sliding interface 300 are assembled with the talus ankle surface 202 of the talus implant 200 in contact with the talus face 302 of the sliding interface 300, the relative motion between the talus implant 200 and the sliding interface 300 in a direction perpendicular to the guiding ridges 204a & 204b is prevented.

The sliding interface 300 of an embodiment of the present invention is composed of an unattached mobile structure sliding interface 300 positioned between the tibial ankle surface 102 and the talus ankle surface 202 in a sandwiched manner having a surface tibia face 301 of the sliding interface 300 complementing the tibial ankle surface 102 of the tibia implant 100, a surface talus face 302 of the sliding interface 300 having at least one negative impression of the guiding ridges 204 of the talus implant 200 as a guiding groove 303 and a negative impression of the talus stopper ridge 205 of the talus implant 200 as a stopper groove 304.

The pre-surgery planning for the implantation of the apparatus for Total Ankle Replacement, firstly a radiographic scan of the patient is carried out. In the preferred embodiment of the present invention, a Computed tomography (CT) scan is performed. While carrying out the said CT scan, the ankle is kept perpendicular to the tibia bone. After CT scan image is generated, bone (tibia, talus, calcaneus etc.) are segmented by using a software accompanying the present invention. The said software is meant for the preop planning and designing. The CT scan image of the ankle is fed into the software for preop planning and designing.

To begin, the range of motion to be carried out by the system is determined. The key movements of the ankle joint complex are plantar-and dorsiflexion, occurring in the sagittal plane; abduction/adduction occurring in the transverse plane and inversion-eversion, occurring in the frontal plane. Combinations of these motions across both the subtalar and tibiotalar joints create three-dimensional motions called supination and pronation. Both terms define the position of the plantar surface of the foot. During supination, a combination of plantarflexion, inversion and adduction causes the sole to face medially. In pronation, dorsiflexion, eversion and abduction act to position the sole facing laterally.

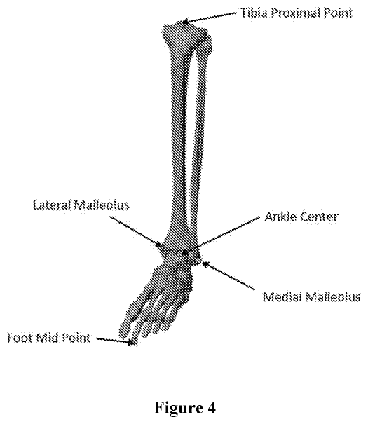

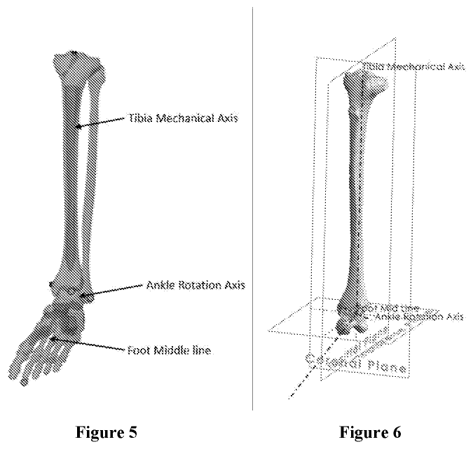

The method implemented by the accompanying software involves, first, picking landmarks on bone like tibia centre, ankle centre, medial malleolus & lateral malleolus, etc, as shown in FIG. 4. Then, creating the required axes including tibia mech axis, ankle rotational axis, foot line, etc, as shown in FIG. 5. According to the said axes, creating the plane of coronal, sagittal and transverse plane, as shown FIG. 6 and measuring the deformities in particular planes, like the illustrated coronal plane and sagittal plane deformity. According to the measured deformities, deciding the cut for tibia and proximal cut for talus. Then implant is placed onto the resected bone.

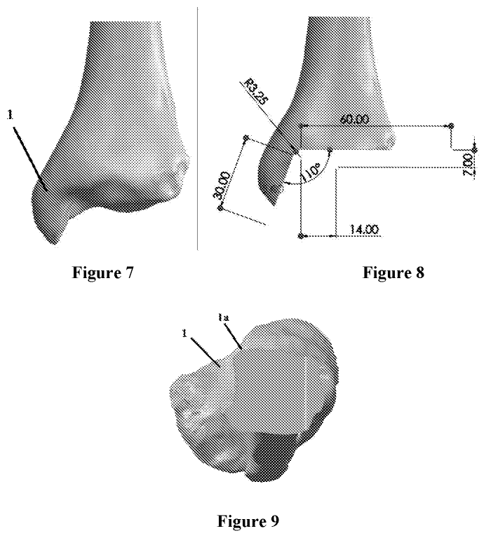

Firstly, the tibia bone is prepared. The FIG. 7 illustrates a tibia bone with a deformity. According to deformity, by applying flexion on the distal plane is defined. After finalizing the distal plane, a plane parallel to the distal plane at most deep side of tibial distal bone is created. Then, another resection is created at some distance. In an exemplary embodiment, a 7 mm cut of tibia bone is created from most tibia deep point. The said plan for the resection of the tibia is illustrated in the FIG. 8. In the said illustration the linear measurements are taken from a predetermined reference point with respect to the subject tibia bone.

It is made sure that no sharp angular cuts are formed during the resection so as to avoid concentration of stress and the resulting vulnerability. As shown, the corner produced is provided a 3.25 mm radius.

According to the resection, patient specific edge curvature is measured and the said curvature is recreated on the sides of the tibia implant 100. So, tibia implant 100 matches the curvature of resected tibia bone. FIG. 9 shows distal view of tibia resection with a curvature which is based on profile of the implant.

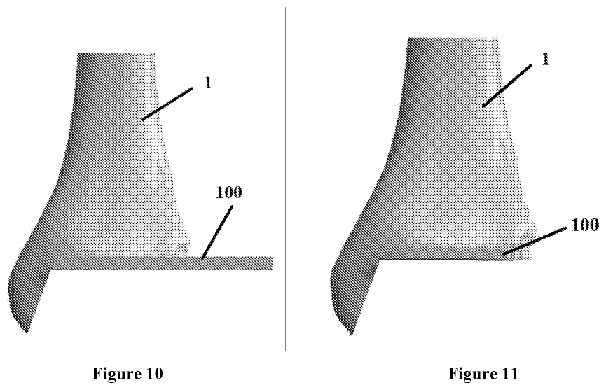

Further, the tibia implant 100 is fixed with tibial bone, as shown in FIG. 10, by providing appropriate constrains.

On the medial side implant has a radius to reduce stress concentration. The thickness of said tibia implant 100 is selected to be 3 mm in the present embodiment of the present invention. Once the tibia implant 100 position is fixed, the outer border of tibia implant 100 is cut such that it matches with tibial bone boundary, as shown in FIG. 11. The position of the tibia is stabilised with respect to the angularly tibia peg 103s provided on the tibial mating surface 101. In current embodiment, the cross-sectional diameter of the tibia peg 103s is 6.5 mm. The length and angle of tibia peg 103 is selected in accordance with the characteristics of the tibia bone of the patient.

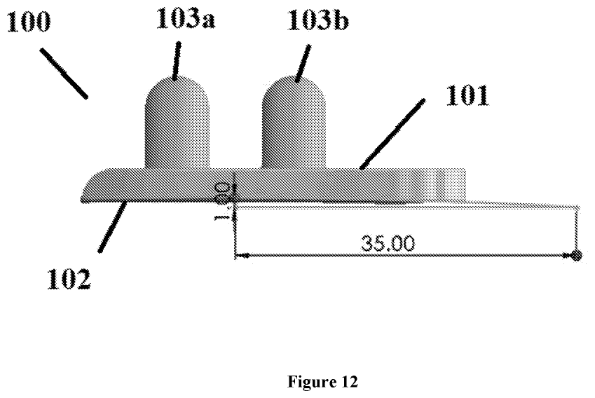

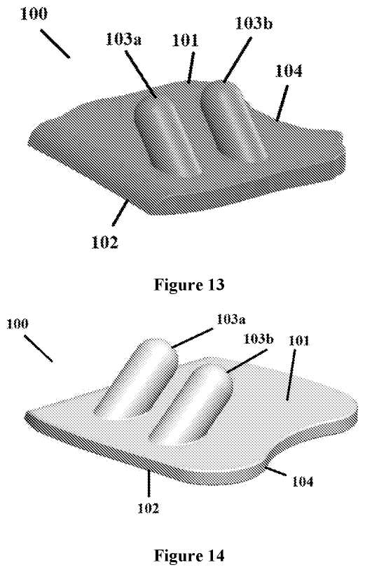

The FIG. 12 illustrates another embodiment of the present invention where the tibial ankle surface 102 is provided with a slight curvature, i.e., with a radius of 35 mm. In the preferred embodiment of the present invention no curvature is provided to the tibial ankle surface 102. Finally, all the sharp edges of the tibia implant 100 are smoothened, as illustrated in the FIG. 13.

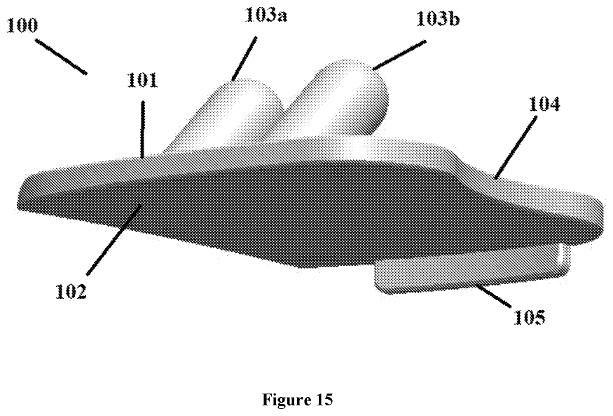

The FIG. 14 and the FIG. 15 illustrate the end form of the tibia implant 100 having a customised tibial peripheral edge 104 which is specific to each patient. The bottom surface is the tibial ankle surface 102 which is to contact the sliding interface 300. The rear edge of the tibial ankle surface 102 is provided with a raised lip i.e., the tibia stopper ridge 105 which prevents the dislocation of the sliding interface 300. The top surface of the tibia implant 100, i.e., the tibial mating surface 101 is to be in joining with the resected tibia bone. The said joining is accomplished by the two angular projections tibia pegs 103a & 103b which are inserted into the resected end of the tibia bone.

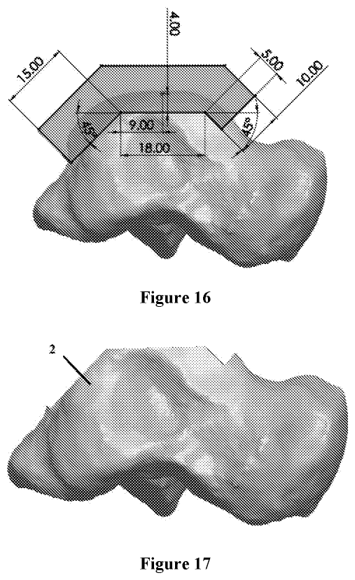

In the next stage, the talus implant 200 is prepared for the talus bone having a deformity. Firstly, a resection is made according to the deformity measured from the CT scan fed into the accompanying software. As illustrated in the FIG. 16 using sagitta plane, a plan is created which includes 3 cuts-proximal cut & Anterior-Posterior Chamfer cut. Depth of the cut is decided specific to the patient. In this exemplary embodiment, a depth of 4 mm is considered. Anterior-posterior chamfer is selected to be at an exemplary 45° angle. In the said illustration the linear measurements are taken from a predetermined reference point with respect to the subject talus bone. The FIG. 17 illustrates a talus bone which has been resected as per the plant illustrated in the FIG. 16.

In accordance with the talus resection plan defined by the FIG. 16, the talus mating surface 201 has contour or profile complementary to the profile created on the talus bone as per the said resection plan. Therefore, the talus mating surface 201 is a continuous surface composed of three conjoined planes, wherein a first proximal horizontal plane 201a is flanked by two planes, one each on the anterior side and the posterior side. The said anterior plane 201a and posterior plane 201b are at an angle with respect to the proximal horizontal plane 201a. In the preferred embodiment of the present invention, the angle that the anterior plane 201b and the posterior plane make with respect to the horizontal plane is an acute angle, i.e. less than 90°, preferably in the range 15°-80°. In the illustrated preferred embodiment the said angle is selected to be 45°. As the proximal horizontal plane 201a is flanked at the anterior and the posterior ends by angularly positioned planes each being at an acute angle with respect to the horizontal plane, the resulting talus mating surface has an overall convex surface. The said angle is selected so as to minimise the magnitude of the talus bone to be resected while ensuring that enough surface area is available for mating with the talus implant 200 in a sturdy manner.

In the preferred embodiment of the present invention, the height of the talus stopper ridge 205 is higher than the height of the two guiding ridges 204a & 204b. The guiding ridges are provided with a shorter height and a rounded profile so as to minimise the wear and tear of the sliding interface 300 from the frequent sliding motion in day-to-day movements. Furthermore, the talus stopper ridge 205 is relatively taller so as to provide greater safety against the failure of the apparatus by the dislocation of the sliding interface 300. The taller height of the talus stopper ridge 205 compensates for the rounded profile by increasing the cross-sectional area of the said talus stopper ridge 205.

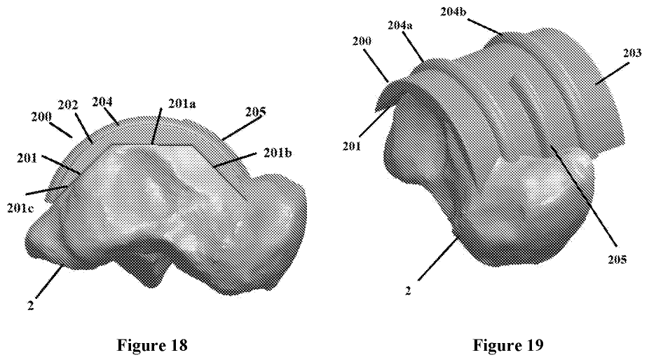

The FIG. 18 illustrates a side view of the talus implant 200 affixed on the resected talus bone. Further, the FIG. 19 illustrates an isometric view of the said talus implant 200 affixed onto the resected talus bone. In the illustrated embodiment of the present invention, on the talus ankle surface 202 of the talus implant 200 two guiding ridges 204a & 204b are provided which are positioned symmetrically and parallel to each other along the entire curved length of the talus ankle surface 202 of the talus implant 200.

In the middle of the said two guiding ridges 204a & 204b, the talus stopper ridge 205 is provided which is parallel to the both said guiding ridges 204a & 204b and is equidistant from the said two guiding ridges 204a & 204b.

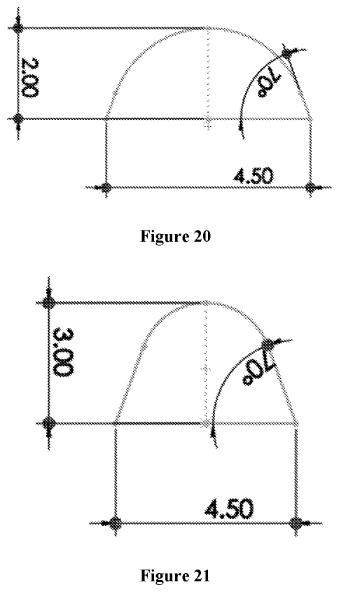

In the preferred embodiment of the present invention, the guiding ridges 204a & 204b on the talus ankle surface 202 are created as per the profile illustrated in the FIG. 20. The cross-sectional profile of the guiding ridges 204a & 204b is defined by a proximal circularly curved section having a radius of 2 mm which is flanked by two straight sections wherein each of the said straight section is at an internal angle of 70°with respect to the talus ankle surface 202. The width of the base of the guiding ridge 204 is 4.5 mm. The circular proximal part of the cross-sectional profile of the guiding ridges 204a & 204b reduces the wear and tear of the sliding interface generated by the frictional forces generated during the relative motion between the sliding interface 300 and the talus implant 200. The proximal circularly curved section of the guiding ridges 204a & 204b may have a radius in the range 1 mm to 3.5 mm for optimum strength against bending and compressive forces. The said internal angle of the straight sections of the cross-sectional profile of the guiding ridges 204a & 204b is selected to be in the range 60°-80° for optimum strength of the said guiding ridges.

The FIG. 21 illustrates the profile according to which the talus stopper ridge 205 is created. The cross-sectional profile of the talus stopper ridge 205 is defined by a proximal circularly curved section having a radius of 3 mm which is flanked by two straight sections wherein each of the said straight sections is at an internal angle of 70° with respect to the talus ankle surface 202. The width of the base of the talus stopper ridge 205 is 4.5 mm. The length of the talus stopper ridge 205 is selected specific to the patient. In the preferred embodiment of the present invention, the length of the talus stopper ridge 205 is less than the length of the guiding ridges 204a & 204b. The circular proximal part of the cross-sectional profile of the talus stopper ridge 205 reduces the wear and tear of the sliding interface generated by the frictional forces generated during the relative motion between the sliding interface 300 and the talus implant 200. The proximal circularly curved section of the talus stopper ridge 205 may have a radius in the range 1.5 mm to 4 mm for optimum strength against bending and compressive forces. The said internal angle of the straight sections of the cross-sectional profile of the talus stopper ridge 205 is selected to be in the range 60°-80° for optimum strength of the said guiding ridges.

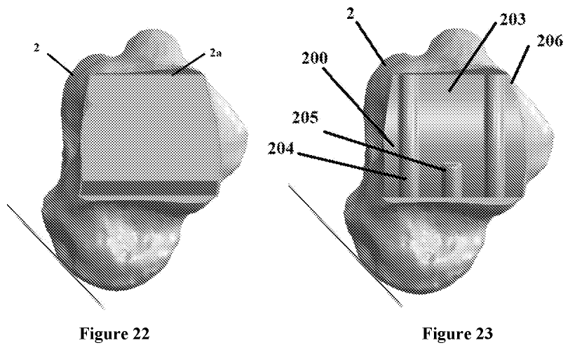

Further, as illustrated in the FIG. 22 the edges of the talus implant 200 are cut to the match the peripheral profile 2a of the cross-section of the resected talus bone 2. The FIG. 23 illustrates the talus implant 200 affixed to the resected talus bone wherein the edges of the said talus implant 200 coincide perfectly coincide with the edges of the cross-sectional periphery of the resected talus bone. The said customised talus peripheral edge 206 is customised specifically as per a particular patient so as to avoid overhanging material which could cause impingement.

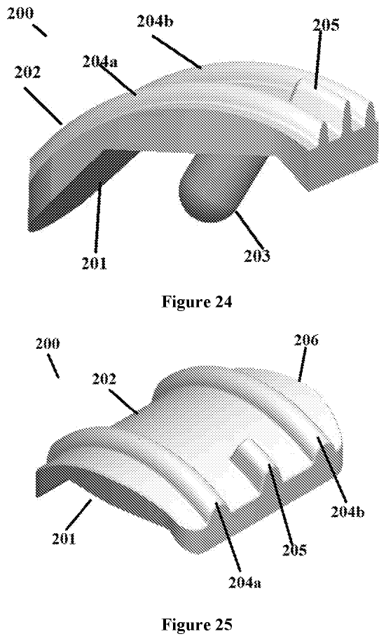

The FIG. 24 illustrates the talus implant 200 where the upper surface, i.e., the talus ankle surface 202 is provided with the guiding ridges 204a & 204b and the talus stopper ridge 205 and the lower surface is provided with an angularly places projection, the talus peg 203. The talus peg 203 is inserted into the resected talus bone so as to stabilise the talus implant 200 on the resected talus bone. The FIG. 25 illustrates the top surface, i.e., the talus ankle surface 202 which contains the two guiding ridges 204a & 204b and the talus stopper ridge 205.

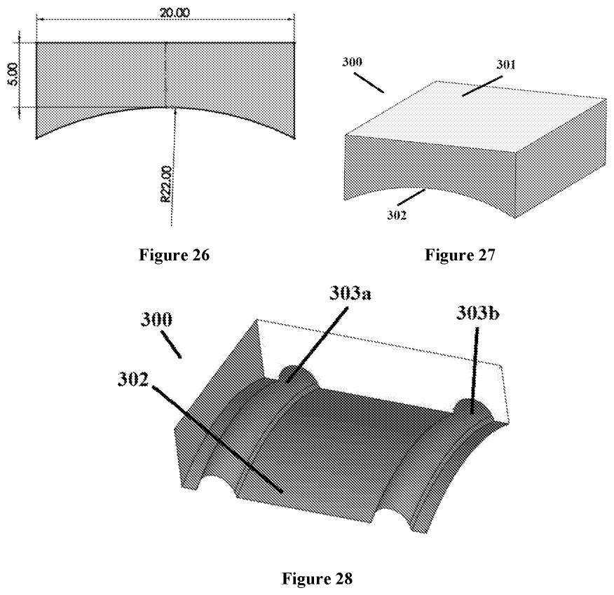

Once the tibia implant 100 and the talus implant 200 are ready, the next step involves the preparation of the sliding interface 300 which is to be located between the said tibia implant 100 and the talus implant 200. To design the sliding interface 300, same resection plane of talus is used. The profile is created as illustrated the in the FIG. 26. In the profile shown whose upper line is parallel to distal plane of talus and bottom curvature is same as talus upper curvature (sketch creating at stoppage plane of talus implant 200). Further, thickness is applied on profile, as shown in FIG. 27, in accordance with the specific patient.

In order to create the talus face 302, i.e., the surface of the sliding interface 300 which is to be in contact with the talus ankle surface 202 of the talus implant 200, the bottom surface of the sliding interface 300 is provided with the same curvature as the talus ankle surface 202. This helps in achieving proper plantar and dorsi flexion motion between talus implant 200 and sliding interface 300.

As illustrated in the FIG. 28, in the curved talus face 302 of the sliding interface 300 two guiding grooves 303a & 303b are cut. The said guiding grooves 303a & 303b are negative impression of the guiding ridges 204a & 204b of the talus implant 200. The said guiding grooves 303a & 303b are meant to mate with the guiding ridges 204a & 204b of the talus implant 200, therefore the said guiding groove have a profile complementary to the guiding ridges 204a & 204b and the location of the said guiding grooves 303a & 303b is such that they perfectly coincide with the guiding ridges 204a & 204b of the talus implant 200.

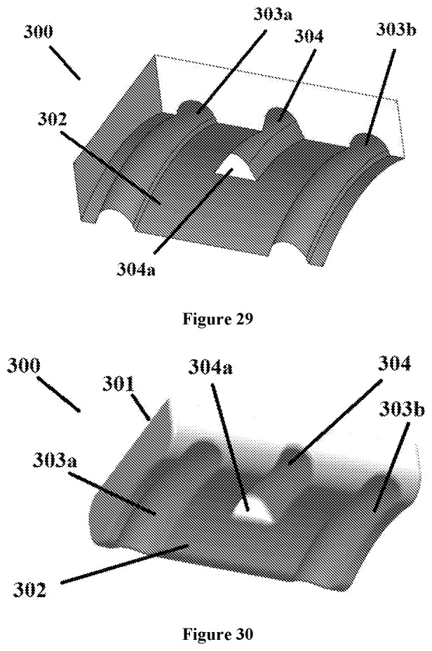

Further, as shown in the FIG. 29 similarly a stopper groove 304 is cut into the talus face 302 of the sliding interface 300 to mate with the talus stopper. The said stopper groove 304 is the negative impression of the talus stopper ridge 205 of the talus implant 200. Therefore, the said stopper groove 304 has a location and profile complementary to the talus stopper groove 304 of the talus implant 200.

Finally, all the sharp corners from the sliding interface 300 are filleted so as to remove the sharp edges and render all the said corners blunt. The resulting sliding interface 300 has been illustrated in the FIG. 30.

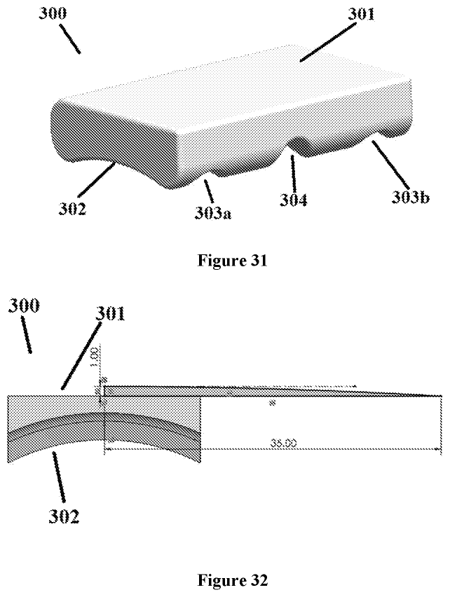

The FIG. 31 illustrates the tibia face 301 of the sliding interface 300, i.e., the surface of the sliding interface 300 is to be in contact with the tibial ankle surface 102 of the tibia implant 100. Since, in the preferred embodiment of the present invention, the tibial ankle surface 102 of the tibia implant 100 is completely flat, therefore tibia face 301 of the sliding interface 300 being complementary to the said tibial ankle surface 102 as well is flat.

The FIG. 32 illustrates the sliding interface 300 of an embodiment of the present invention where the tibial ankle surface 102 of the tibia implant 100 is provided with a concave curvature having a radius of 35 mm. Since the tibia face 301 of the sliding interface 300 is required to be complementary to the said tibial ankle surface 102, the said tibia face 301 is provided with a convex curvature having a radius of 35 mm.

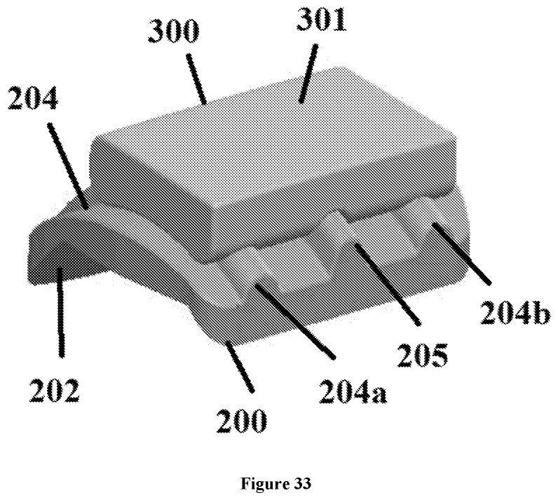

The FIG. 33 illustrates the assembly of the talus implant 200 and the sliding interface 300 where the guiding ridges 204a & 204b and the talus stopper ridge 205 are mated with the complementary guiding grooves 303a & 303b and the stopper groove 304 of the sliding interface 300 thus locking them against lateral relative movement while allowing the relative sliding motion in the direction of the guiding ridges 204a & 204b. The talus stopper ridge 205 along with the stopper groove 304 prevent the relative sliding motion of the talus implant 200 and the sliding interface 300 is limited so as to prevent the dislocation of the sliding interface 300. The said relative sliding motion between the talus implant 200 and the sliding interface 300 is only allowed until the talus stopper ridge 205 of the talus implant 200 is stopped by the stopper groove end 304a of the sliding interface 300.

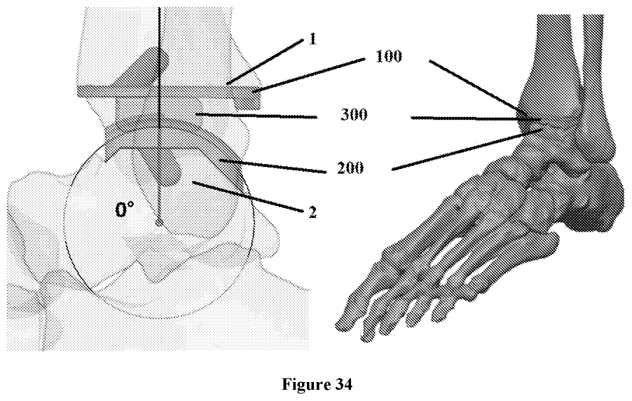

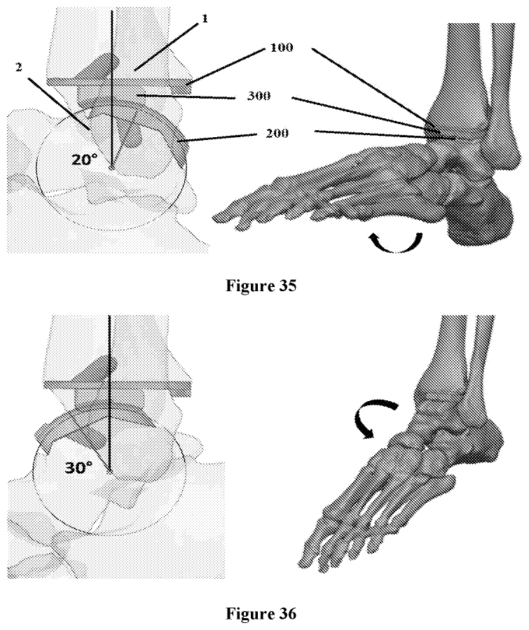

The FIG. 34 illustrates an ankle implanted with the apparatus for total ankle replacement. In the said figure, the implanted angle is in normal position, i.e., there is no dorsiflexion or plantar flexion movement taken place between the tibia bone and the talus bone. The FIG. 35 an implanted angle undergoing dorsiflexion where there is relative displacement between talus implant 200 and the sliding interface 300. When the talus bone is rotated with respect to the tibia bone, the talus implant 200 slides along the path defined by the mating of the guiding ridges 204a & 204b on the talus ankle surface 202 and the complementary guiding grooves 303a & 303b on the talus face 302 of the sliding interface 300. The stopper ridge of the talus implant 200 in conjunction with the stopper groove 304 of the sliding interface 300 prevent the dislocation of the sliding interface 300 from between the tibia implant 100 and the talus implant 200. The illustrated embodiment of the present invention allows up to 20°of dorsiflexion of the ankle joint.

The FIG. 36 illustrates an ankle joint implanted with the apparatus or total ankle replacement. In the said illustration, the implanted angle is undergoing plantarflexion i.e., there is relative rotational motion between the tibia bone and the talus bone. The talus bone rotates forward along the path defined by the mating of the guiding ridges 204a & 204b on the talus ankle surface 202 and the complementary guiding grooves 303a & 303b on the talus face 302 of the sliding interface 300. The illustrated embodiment of the present invention allows up to 20° of plantarflexion of the ankle joint. Therefore a total range of rotational motion achieved by the illustrated embodiment of the present invention is 50° when 20° of dorsiflexion and 30° of plantarflexion are considered. In comparison the widely implemented total ankle systems of the prior art including the Hintermann's and STAR Ankle's provided an inferior total range of motion in the range 35°-45°. Therefore, the present invention provides an improvement of at least 11% in the range of motion over the some of the most popular devices of the prior art In the preferred embodiment of the present invention, the tibia implant 100 and the talus implant 200 are made from a medical grade material such as Titanium alloys, cobalt-chromium alloys, tantalum etc. In the preferred embodiment, the sliding interface 300 is made from ultra-high molecular weight polyethylene (UHMWPE). The said tibia implant 100 and the talus implant 200 may further be provided with a titanium nitride (TiN) coating to enhance biocompatibility.

Furthermore, the tibial mating surface 101 of the tibia implant 100 and the talus mating surface 201 of the talus implant 200 is provided with a surface roughness to enhance osseointegration, i.e., bone ingrowth into a metal implant. Preferably, the surface roughness of the said tibial mating surface 101 and the talus implant 200 surface are kept in the range 1 μm-5 μm.

As the apparatus for total ankle replacement is an implant for the body, the said apparatus is provided with anti-infective coating to prevent the risk of infection arising from the site of implant. Further, the apparatus is also provided with immune-evasive coating which prevents a reaction from the immune system of the body when the present invention is implanted into the body at the respective fracture site.

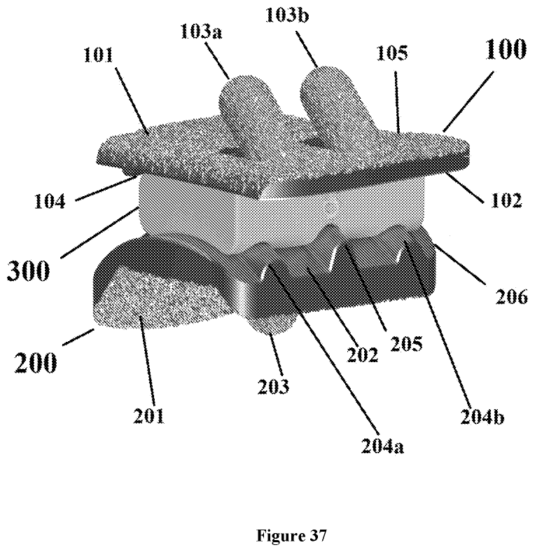

The FIG. 37 illustrates an assembled form of a preferred embodiment of the apparatus for total ankle replacement having dual-mobility by implementing a three-component system comprising a tibia implant 100 for the tibia bone, a talus implant 200 for the talus bone and a sliding interface 300 sandwiched between the said tibia implant 100 and the talus implant 100 in a unattached or mobile manner. As the sliding interface 300 is not rigidly attached to either the tibia implant or the talus implant, a greater magnitude of relative motion is allowed between the tibia implant 100 and the talus implant 200 this a greater range of motion between the tibia bone and the talus bone.

The said figure depicts an embodiment of the implant device for total ankle replacement comprising a tibia implant 100 having at least one angularly positioned cylindrical projection tibia peg 103 on a tibia mating surface 101, a tibial ankle surface 102 of the said tibia implant 100 is provided with a raised lip tibia stopper ridge 105 on its rear edge for the prevention of the dislocation of a sliding interface 300.

A talus implant 200 having at least one angularly positioned cylindrical projection talus peg 203 on a talus mating surface 201, a talus ankle surface 202 is provided with at least one raised projection guiding ridge 204 running along the entire anterior-posterior length of the said talus implant 200 and a raised projection talus stopper ridge 205 running along the partial anterior-posterior length of the said talus implant 200.

An unattached mobile structure sliding interface 300 positioned between the tibial ankle surface 102 and the talus ankle surface 202 in a sandwiched manner having a surface tibia face 301 of the sliding interface 300 complementing the tibial ankle surface 102 of the tibia implant 100, a surface talus face 302 of the sliding interface 300 having at least one negative impression of the guiding ridges 204 of the talus implant 200 as a guiding groove 303 and a negative impression of the talus stopper ridge 205 of the talus implant 200 as a stopper groove 304.

The tibial ankle surface 102 of the talus implant 100 has an overall flat profile. The talus ankle surface 202 of the talus implant 200 has an overall convex profile. The tibia face 301 has an overall flat profile in order to be complementary to the tibia ankle surface 102 of the tibia implant 100. The talus face 302 has an overall concave profile in order to be complementary to the talus ankle surface 202 of the talus implant 200. The peripheral edge of the tibia implant 100 is a customised tibial peripheral edge 104 having a personalised profile to match the cross-sectional edge of the resected tibia bone. The peripheral edge of the talus implant 200 is a customised talus peripheral edge 206 having a personalised profile to match the cross-sectional edge of the resected talus bone. The overall concave profile of the talus mating surface 201 of the talus implant 200 is composed of a proximal horizontal plane 201a to which an anterior plane 201b and a posterior plane 201c joined at the anterior end and the posterior end of the said proximal plane respectively with the said anterior plane and the posterior plane forming an acute angle with respect to the horizontal proximal plane. The tibia implant 100 is made from a material selected from Titanium alloys, cobalt-chromium alloys, tantalum. The talus implant 200 is made from a material selected from Titanium alloys, cobalt-chromium alloys, tantalum. The sliding interface 300 is made from ultra-high molecular weight polyethylene. All the edges of the tibia implant 100, talus implant 200 and the sliding interface 300 are filleted. The cross-sectional profile of the guiding ridges 204a & 204b is defined by a proximal circularly curved section having a radius in the range 1 mm to 3.5 mm which is flanked by two straight sections wherein each of the said straight section is at an internal angle in the range of 60°-80° with respect to the talus ankle surface 202. The cross-sectional profile of the talus stopper ridge 205 is defined by a proximal circularly curved section having a radius of 1.5 mm to 4 mm which is flanked by two straight sections wherein each of the said straight sections is at an internal angle in the range of 60°-80° with respect to the talus ankle surface 202. The tibial mating surface 101 of the tibia implant 100 is provided with a surface roughness in the range 1 μm-5 μm. The talus mating surface 201 of the talus implant 100 is provided with a surface roughness in the range 1 μm-5 μm. The height of the talus stopper ridge 205 is greater than the height of the guiding ridges 204a & 204b. The tibia implant 100 and the talus implant 200 are provided with a titanium nitride (TIN) coating for biocompatibility. The thickness of the tibia stopper ridge 105 is in the range 0.8 mm to 2.5 mm.

1. An implant device for total ankle replacement comprising:
a tibia implant 100 having at least one angularly positioned cylindrical projection tibia peg 103 on a tibia mating surface 101, a tibial ankle surface 102 of the said tibia implant 100 is provided with a raised lip tibia stopper ridge 105 on its rear edge for the prevention of the dislocation of a sliding interface 300;
a talus implant 200 having at least one angularly positioned cylindrical projection talus peg 203 on a talus mating surface 201, a talus ankle surface 202 is provided with at least one raised projection guiding ridge 204 running along the entire anterior-posterior length of the said talus implant 200 and a raised projection talus stopper ridge 205 running along the partial anterior-posterior length of the said talus implant 200;
an unattached mobile structure sliding interface 300 positioned between the tibial ankle surface 102 and the talus ankle surface 202 in a sandwiched manner having a surface tibia face 301 of the sliding interface 300 complementing the tibial ankle surface 102 of the tibia implant 100, a surface talus face 302 of the sliding interface 300 having at least one negative impression of the guiding ridges 204 of the talus implant 200 as a guiding groove 303 and a negative impression of the talus stopper ridge 205 of the talus implant 200 as a stopper groove 304.

2. The implant device for total ankle replacement, as claimed in claim 1, wherein the tibial ankle surface 102 of the talus implant 100 has an overall flat profile.

3. The implant device for total ankle replacement, as claimed in claim 1, wherein the talus ankle surface 202 of the talus implant 200 has an overall convex profile.

4. The implant device for total ankle replacement, as claimed in claim 1, wherein the tibia face 301 has an overall flat profile in order to be complementary to the tibia ankle surface 102 of the tibia implant 100.

5. The implant device for total ankle replacement, as claimed in claim 1, wherein the talus face 302 has an overall concave profile in order to be complementary to the talus ankle surface 202 of the talus implant 200.

6. The implant device for total ankle replacement, as claimed in claim 1, wherein the peripheral edge of the tibia implant 100 is a customised tibial peripheral edge 104 having a personalised profile to match the cross-sectional edge of the resected tibia bone.

7. The implant device for total ankle replacement, as claimed in claim 1, wherein the peripheral edge of the talus implant 200 is a customised talus peripheral edge 206 having a personalised profile to match the cross-sectional edge of the resected talus bone.

8. The implant device for total ankle replacement, as claimed in claim 1, wherein the overall concave profile of the talus mating surface 201 of the talus implant 200 is composed of a proximal horizontal plane 201a to which an anterior plane 201b and a posterior plane 201c joined at the anterior end and the posterior end of the said proximal plane respectively with the said anterior plane and the posterior plane forming an acute angle with respect to the horizontal proximal plane ..

9. The implant device for total ankle replacement, as claimed in claim 1, wherein the tibia implant 100 is made from a material selected from Titanium alloys, cobalt-chromium alloys, tantalum.

10. The implant device for total ankle replacement, as claimed in claim 1, wherein the talus implant 200 is made from a material selected from Titanium alloys, cobalt-chromium alloys, tantalum.

11. The implant device for total ankle replacement, as claimed in claim 1, wherein the sliding interface 300 is made from ultra-high molecular weight polyethylene.

12. The apparatus for total ankle replacement, as claimed in claim 1, wherein all the edges of the tibia implant 100, talus implant 200 and the sliding interface 300 are filleted.

13. The implant device for total ankle replacement, as claimed in claim 1, wherein the cross-sectional profile of the guiding ridges 204a & 204b is defined by a proximal circularly curved section having a radius in the range 1 mm to 3.5 mm which is flanked by two straight sections wherein each of the said straight section is at an internal angle in the range of 60°-80°with respect to the talus ankle surface 202.

14. The implant device for total ankle replacement, as claimed in claim 1, wherein the cross-sectional profile of the talus stopper ridge 205 is defined by a proximal circularly curved section having a radius of 1.5 mm to 4 mm which is flanked by two straight sections wherein each of the said straight sections is at an internal angle in the range of 60°-80° with respect to the talus ankle surface 202.

15. The implant device for total ankle replacement, as claimed in claim 1, wherein the tibial mating surface 101 of the tibia implant 100 is provided with a surface roughness in the range 1 μm-5 μm.

16. The implant device for total ankle replacement, as claimed in claim 1, wherein the talus mating surface 201 of the talus implant 100 is provided with a surface roughness in the range 1 μm-5 μm.

17. The implant device for total ankle replacement, as claimed in claim 1, wherein the height of the talus stopper ridge 205 is greater than the height of the guiding ridges 204a & 204b.

18. The implant device for total ankle replacement, as claimed in claim 1, wherein the tibia implant 100 and the talus implant 200 are provided with a titanium nitride (TiN) coating for biocompatibility.

19. The implant device for total ankle replacement, as claimed in claim 1, wherein the thickness of the tibia stopper ridge 105 is in the range 0.8 mm to 2.5 mm.
